(19) United States
(12) Patent Application Publication
(10) Pub. No.: US 2007/0136837 A1
(43) Pub. Date: Jun. 14, 2007

(54) Glyphosate tolerant plants and methods of making and using the same

(75) Inventors: Calvin F. Konzak, Seattle, WA (US); Thomas B. Rice, Henderson, NV (US)

Correspondence Address: SCHWEGMAN, LUNDBERG, WOESSNER & KLUTH, P.A., P.O. BOX 2938, MINNEAPOLIS, MN, 55402, US

(73) Assignee: OMEGA GENETICS, LLC, HENDERSON, NV (US)

(21) Appl. No.: 11/490,855

(22) Filed: Jul. 21, 2006

Related U.S. Application Data
(63) Continuation of application No. PCT/US05/01568, filed on Jan. 21, 2005.
(60) Provisional application No. 60/538,050, filed on Jan. 21, 2004.
(60) Provisional application No. 60/603,420, filed on Aug. 20, 2004.

(51) Int. Cl. A01H 1/00; C12N 15/82; A01H 5/00; C12N 5/04

(52) U.S. Cl. 800/278; 800/320.3; 435/468; 435/419

(57) ABSTRACT

A crop plant having an induced mutant allele of an endogenous gene, the induced mutant allele conferring tolerance to glyphosate as compared with a wild-type plant of the same species, wherein the glyphosate tolerance is due to the presence of the induced mutant allele of the endogenous plant gene, is provided. Also provided are methods of making and using the crop plant.

This application claims the benefit of U.S. Provisional Patent Application No. 60/538,050, filed Jan. 21, 2004, and No. 60/603,420, filed Aug. 20, 2004, the disclosures of which are incorporated by reference herein.

### BACKGROUND OF THE INVENTION

Weed species have long been a problem in cultivated fields. Although once a labor intensive operation, weed control has been made easier by the availability of efficient weed killing chemical herbicides. The wide-spread use of herbicides, along with improved crop varieties and fertilizers, has made a significant contribution to the “green revolution” in agriculture. Particularly useful herbicides are those that have a broad spectrum of herbicidal activity. Unfortunately, broad spectrum herbicides typically have a deleterious effect on crop plants exposed to the herbicide. One way to overcome this problem is to produce plants that are tolerant to certain broad spectrum herbicides.

One particular broad spectrum herbicide that has been the subject of much investigation is N-phosphonomethyl-glycine, also known as glyphosate. Glyphosate has been used extensively by farmers world wide for controlling weeds prior to crop planting, for example, in no-till farming. In addition, glyphosate is an efficient means to control weeds and volunteer plants between production cycles or crop rotations. Glyphosate does not carry-over in soils after use, and it is widely considered to be one of the most environmentally safe and broadly effective of chemical herbicides available for use in agriculture.

Glyphosate kills plants by inhibiting the shikimic acid pathway. This pathway leads to the biosynthesis of aromatic compounds, including amino acids, vitamins and plant hormones. Glyphosate blocks the conversion of phosphoenolpyruvic acid (PEP) and 3-phosphoshilkimic acid to 5-enolpyruvyl-3-phosphoshikimic acid by binding to and inhibiting activity of the enzyme 3-enolpyruvylshikimate-3-phosphate synthase, commonly referred to as EPSP synthase, or EPSPS.

Unfortunately, no crop plants are known that are naturally tolerant to glyphosate and therefore the utility of this herbicide for weed control in cultivated crops has been limited. One method to produce glyphosate tolerant crop plants is to introduce a gene encoding a heterologous glyphosate tolerant form of an EPSPS gene into the crop plant using the techniques of genetic engineering. Using chemical mutagenesis, glyphosate tolerant forms of EPSPS were produced in bacteria and the heterologous genes were introduced into plants to produce glyphosate tolerant plants (see, e.g., Comai et al., Science 221:370-71 (1983)). The heterologous EPSPS genes are usually overexpressed in the crop plants to obtain the desired level of tolerance.

Tolerance to glyphosate in bacterial genes has been reported to be due to alterations in the EPSPS amino acid sequence (see, e.g., Stalker et al., J. Biol. Chem. 260:4724-28 (1985)). Amino acid substitutions are believed to change the enzyme structure sufficiently to reduce binding of glyphosate to the enzyme. The altered enzyme retains sufficient biosynthetic activity for plant growth and development, but is tolerant to inhibition by glyphosate.

In addition, a bacterial species, Agrobacterium strain CP4 (see U.S. Pat. No. 5,627,061) was identified that is naturally tolerant to glyphosate. A gene encoding a glyphosate tolerant form of EPSPS was cloned from this species and subsequently introduced into plants, including maize (see EP 1167531) and wheat (see U.S. Pat. No. 6,689,880), using genetic engineering techniques. The resulting genetically-modified plants are tolerant to field applications of glyphosate. Although glyphosate tolerant crop plants have been produced using genetic engineering techniques, commercial acceptance of such crops has been hindered by wide spread resistance to genetically modified organisms (GMO) as food sources.

In theory, a second method to produce glyphosate tolerant crop plants is to alter the endogenous glyphosate-sensitive EPSPS gene by mutagenesis, thereby producing glyphosate tolerant crop plants without using the techniques of genetic engineering. A difficulty with mutagenesis technology, as applied to higher eukaryotic plants, is inducing tolerance-conferring mutations in a sufficient number of target genes to obtain the desired phenotype. Each haploid genome may carry more than one target gene (e.g., in the case of multigene families). A polyploid state increases the number of potential gene targets. Many plant species are functional polyploids or passed through a polyploid stage during their evolution. Without knowledge of the relative contributions of each copy of the target gene to the amount of EPSPS enzyme produced, there has been uncertainty as to whether specific herbicide tolerance mutations can be induced and the number of mutated genes that would be required to the confer the desired herbicide tolerance phenotype.

Previous attempts to develop glyphosate tolerant plants by modification of the endogenous EPSPS gene(s) have met with limited success. Glyphosate tolerant cell cultures of carrot (Shyr et al., Mol. Gen. Genet. 232: 377-82 (1992)) as well as alfalfa, soybean and tobacco (Widholm et al., Physiol. Plant. 112: 540-45 (2001)) were produced. In all instances glyphosate tolerance was the result of increased expression of the endogenous EPSPS gene by gene amplification, not alteration of the amino acid sequence of the endogenous EPSPS enzyme. No plants were reported produced from these gene-amplified glyphosate tolerant cell lines. There is, therefore, no suggestion as to whether glyphosate tolerance due to gene amplification would be maintained in these plants. More importantly, there is no suggestion as to whether glyphosate tolerance due to gene amplification would be genetically stable in an intact plant and inherited by progeny plants.

The difficulty of isolating glyphosate tolerant plants through modification of an endogenous EPSPS gene has been further addressed in Arabidopsis. M2 progeny of ethylmethanesulfonate (EMS) mutagenized Arabidopsis lines were screened for resistance to glyphosate, imidazolinone or sulfonylurea herbicides (Jander et al, Plant. Physiol. 131:139-46 (2003)). No glyphosate tolerant mutant plants were identified among M(2) progeny of 125,000 Columbia and 125,000 Landsberg erecta M(1) lines. Mutant plants tolerant to both imidazolinone and sulfonylurea herbicides were isolated. It was estimated that in these mutant populations, screening of fewer than 50,000 M1 lines would suffice to give a 95% probability of finding a mutation in any G:C base pair in the Arabidopsis genome. These results emphasize the great difficulty in producing mutations in plants conferring glyphosate resistance in the endogenous EPSPS gene as compared to producing mutants tolerant to other herbicides.

### BRIEF SUMMARY OF THE INVENTION

The present invention provides glyphosate tolerant plants having one or more induced mutant allele(s) of an endogenous plant gene(s), the mutant allele(s) conferring glyphosate tolerance. Also provided are plant parts, plant cells and seeds from the glyphosate tolerant plants described herein. Further provided are methods for inducing and isolating glyphosate tolerance mutant alleles in target plants, methods for recovering induced mutant alleles conferring glyphosate tolerance, methods for further increasing the level of glyphosate tolerance, methods for transferring the induced mutant alleles conferring glyphosate tolerance to other varieties, and methods for controlling weeds in the vicinity of crop plants.

In one aspect, a plant comprising an induced mutant allele(s) of an endogenous gene(s) is provided. The induced mutant allele(s) confers tolerance to glyphosate as compared with a wild-type (or “normal”) plant of the same species or variety. The glyphosate tolerance of the plant is due to the presence of the induced mutant allele(s) of the endogenous plant gene(s). In some embodiments, the plant is free of recombinant glyphosate tolerance genes.

In some embodiments, the plant is a crop plant (e.g., agronomic, vegetable, turf grass, horticultural plant, or the like). The plant can be, for example, alfalfa, beans, bent grass, bermuda grass, blue grass, brome grass, cereal, carrot, chickpea, cotton, cowpea, cucumbers, dwarf bean, fescue, field bean, flax, forage grasses, garlic, kenaf, lima bean, lupini bean, oilseed rape, onion, peas, peanut, peppers, pigeon pea, pineapple, potato, ryegrass, soybean, squash, sugar beets, sunflower, tomato, or the like. The cereal can be, for example, barley, corn, millet, oats, rice, rye, sorghum, triticale, wheat, or the like. The wheat can be, for example, a bread wheat or durum wheat.

The plant can be tolerant to a dosage of, for example, about 8 oz per acre, about 16 oz per acre, about 24 oz per acre, about 32 oz per acre, about 40 oz per acre, about 52 oz per acre, or more. In some embodiments, the plant comprises at least two induced mutant alleles of endogenous genes, the induced alleles conferring tolerance to glyphosate.

In related aspects, seeds from glyphosate tolerant plants and glyphosate tolerant progeny plants are provided.

In another aspect, a polyploid plant carrying an induced mutant allele(s) of an endogenous gene(s) that confer(s) tolerance to glyphosate is provided. Glyphosate tolerance is due to the presence of the induced mutant allele(s) of the endogenous plant gene or genes. In some embodiments, the polyploid plant is free of recombinant glyphosate tolerance genes.

The glyphosate tolerant polyploid plant can be, for example, a cereal, such as a triticale or wheat plant. The wheat plant, can be, for example, T. aestivum, T. turgidum, T. timopheevii, T. zhukovskyi species or a hybrid thereof. In some embodiments, the wheat is a bread wheat or a durum wheat.

In some embodiments, the polyploid plant can carry at least two different induced mutant alleles in different endogenous genes, each mutation conferring tolerance to glyphosate. The induced mutant alleles can be, for example, in different copies of a gene family or in different genomes. In related aspects, seed and progeny derived from such polyploid plants are provided.

In yet another aspect, a polyploid wheat plant carrying an induced mutant allele(s) of an endogenous wheat gene(s) is provided. The induced mutant allele(s) confer(s) tolerance to glyphosate. In some embodiments, the wheat plant is free of recombinant glyphosate tolerance genes.

The polyploid wheat plant can be tolerant to a dosage of about 8 oz per acre, about 16 oz per acre, about 24 oz per acre, about 32 oz per acre, about 40 oz per acre, about 52 oz per acre, or more. The polyploid wheat plant can be, for example, a bread wheat or a durum wheat.

In some embodiments, the polyploid wheat plant carries at east two different induced mutant alleles in different endogenous wheat genes that confer tolerance to glyphosate. For example, the induced mutant alleles can be in different EPSPS genes in the same or in different wheat genomes. In a related aspect, seed and progeny of such polyploid wheat plants are provided.

In another aspect, a method for inducing glyphosate tolerance mutant alleles in the genome of a plant is provided. The method typically includes providing seed from a target plant; consecutively contacting the seed with an effective amount of at least two mutagenic agents to yield mutagenized seeds; germinating the mutagenized seeds to form M1 mutagenized plants to produce M2 generation seeds; germinating the M2 generation seeds to produce M2 generation plants, applying glyphosate to the M2 generation plants; and screening the M2 generation plants to identify glyphosate tolerant plants.

In some embodiments, glyphosate is applied at a dosage of at least about 8 oz per acre, at least about 16 oz per acre, at least about 24 oz per acre, at least about 32 oz per acre, at least about 40 oz per acre, or least about 52 oz per acre, or more. Glyphosate can be applied, for example, when the M2 progeny plants are at the three to five leaf stage.

In some embodiments, the method includes inducing two different glyphosate tolerance mutant alleles, each in a different gene either within the same genome or in different genomes. The plant can be, for example, a crop plant (e.g., agronomic, vegetable, turf grass or horticultural plant). The plant can be, for example, alfalfa, beans, bent grass, bermuda grass, blue grass, brome grass, cereal, carrot, chickpea, cotton, cowpea, cucumbers, dwarf bean, fescue, field bean, flax, forage grasses, garlic, kenaf, lima bean, lupini bean, oilseed rape, onion, peas, peanut, peppers, pigeon pea, pineapple, potato, ryegrass, soybean, squash, sugar beets, sunflower, tomato, or the like. The cereal can be, for example, barley, corn, millet, oats, rice, rye, sorghum, triticale, wheat, or the like. The wheat can be, for example, a bread wheat or durum wheat.

In another aspect, a method for increasing the level of glyphosate tolerance by inducing additional glyphosate tolerance mutant alleles in the genome of a plant is provided. The method typically includes providing seed from a target plant containing induced glyphosate tolerance mutant allele(s); consecutively contacting the seed with an effective amount of at least two mutagenic agents to yield mutagenized seeds; germinating the mutagenized seeds to form M1 mutagenized plants to produce M2 generation seeds; germinating the M2 generation seeds to produce M2 generation plants, applying glyphosate to the M2 generation plants; and screening the M2 generation plants to identify glyphosate tolerant plants with a higher level of glyphosate tolerance.

In some embodiments, the method can further include crossing the induced glyphosate tolerance mutant allele(s) into a non-glyphosate tolerant plant to form a glyphosate tolerant progeny plant. In related aspects, seed can be obtained from the plants.

In yet another aspect, a method of altering the glyphosate tolerance of a target plant is provided. The method includes crossing a first plant carrying a first glyphosate tolerance mutant allele(s) with the target plant to form a progeny plant having a glyphosate tolerant phenotype, the progeny plant carrying the glyphosate tolerance mutant allele. In some embodiments, the progeny plant is free of recombinant glyphosate tolerance genes. In other embodiments, the target plant carries a recombinant glyphosate tolerance gene.

In some embodiments, the progeny plant can include one or more glyphosate tolerance mutant alleles and a recombinant glyphosate tolerance gene, wherein both the glyphosate tolerance mutant allele(s) and recombinant glyphosate tolerance gene contribute to the glyphosate tolerant phenotype. The glyphosate tolerant phenotype of the progeny plant can be, for example, greater than the glyphosate tolerance phenotype of the target plant. In some embodiments, a plurality of glyphosate tolerance mutant alleles is crossed into the target plant.

In yet another aspect, a method of increasing the level of glyphosate tolerance of a target plant is provided. The method includes crossing a first plant carrying a first glyphosate tolerance mutant allele(s) with a target plant carrying a different glyphosate tolerance mutant allele(s) to form a progeny plant having a higher glyphosate tolerant phenotype than either parent, the progeny plant carrying the glyphosate tolerance mutant alleles of both parents. In some embodiments, the progeny plant is free of recombinant glyphosate tolerance genes. In other embodiments, the target plant carries a recombinant glyphosate tolerance gene.

In yet another aspect, a method of controlling weeds within the vicinity of a crop plant (e.g., plant of an agronomic, horticultural, turf grass, vegetable species or the like) is provided. The method generally includes applying glyphosate to weeds and, optionally, the crop plant, the crop plant comprising an induced mutant allele(s) conferring increased tolerance to the glyphosate as compared to a wild-type variety of the plant. In some embodiments, the crop plant is free of recombinant glyphosate tolerance genes.

In some embodiments, glyphosate is applied at a dosage of at least about 8 oz per acre, at least about 16 oz per acre, at least about 24 oz per acre, at least about 32 oz per acre, at least about 40 oz per acre, at least about 52 oz per acre, or more. The weeds can be, for example, annual grass, biennial grass, perennial grass, broadleaf weeds, and volunteer crop plants; such weeds include, but are not limited to, wild oats, foxtail grasses, quackgrass, pigweed, field bindweed, wild buckwheat, knapweed, cheat grass, Barnyard grass, goat grall, black grass, sweet clover, smartweed, yellow mustard, kochia, cocklebur, velvet leaf, wild sunflower, biennial wormwood, and/or Russian thistle. The weed is susceptible to the dosage of glyphosate applied.

The crop plant (e.g., agronomic, vegetable, turf grass or horticultural plant) can be, for example, alfalfa, beans, bent grass, bermuda grass, blue grass, brome grass, cereal, carrot, chickpea, cotton, cowpea, cucumbers, dwarf bean, fescue, field bean, flax, forage grasses, garlic, kenaf, lima bean, lupini bean, oilseed rape, onion, peas, peanut, peppers, pigeon pea, pineapple, potato, ryegrass, soybean, squash, sugar beets, sunflower, tomato, or the like. The cereal can be, for example, barley, corn, millet, oats, rice, rye, sorghum, triticale, wheat, or the like. The wheat can be, for example, a bread wheat or durum wheat.

### DETAILED DESCRIPTION OF THE INVENTION

The present inventors have surmounted the problem of production of glyphosate tolerant plants without the use of genetic engineering methods. In addition, the present inventors have surmounted the difficulties of mutagenesis technology as applied to plants and specifically as applied to the isolation of glyphosate tolerant plants. Using the methods described herein, glyphosate tolerant plants were produced by mutagenesis technology. Thus, the present invention provides glyphosate tolerant plants having at least one induced mutant allele of an endogenous plant gene that is stably inherited by progeny plants.

The present invention also provides plant parts, plant cells and seeds from the glyphosate tolerant plants described herein. Further provided are methods for inducing and isolating glyphosate tolerance mutant alleles in target plants, methods for recovering induced mutant alleles conferring glyphosate tolerance, methods for further increasing the level of glyphosate tolerance, methods for transferring the induced mutant alleles conferring glyphosate tolerance to other varieties, and methods for controlling weeds in the vicinity of crop plants.

The induced mutations are induced in an endogenous gene in a target plant. An “endogenous gene” refers to a gene normally present in the genome of the plant or which is introduced into the plant by plant breeding techniques. For example, the term “endogenous gene” includes a gene normally present in the plant genome, a gene introduced into a plant (e.g., wheat) by interbreeding different varieties, cultivars, lines or the like, of the same plant species, and by interbreeding related species (e.g., wheat crossed with emmer). The term “endogenous gene” excludes heterologous genes from other genera if such genes cannot be introduced into the target plant by plant breeding techniques (e.g., conventional plant breeding techniques, including techniques for producing ‘wide’ crosses). For example, a bacterial gene introduced by recombinant methodologies is not an endogenous plant gene. The glyphosate tolerance mutant allele is typically present at its normal chromosomal locus and is, for example, an allele of a wild-type gene. In some embodiments, the endogenous gene is an EPSPS gene. In other embodiments, the gene can encode a glyphosate N-acetyltransferase enzyme. When multiple glyphosate tolerance mutant alleles (e.g., multiple alleles of the EPSPS gene) are present in a polyploid plant, they can be located in the same genome or in different genomes. For example, in a tetraploid or hexaploid wheat, glyphosate tolerance mutant alleles can be present in the A, B and/or D genomes, as applicable.

An “induced mutant allele” conferring glyphosate tolerance has a mutation resulting from a mutagenesis technology (see infra) and does not refer to alterations of plant genes by naturally occurring events, such as spontaneous mutations. The latter occurs in the absence of mutagenic treatment. Mutations conferring glyphosate tolerance can be due to, for example, one or more DNA nucleotide insertions, deletions, substitutions (e.g., a transition or transversion), or the like, in an endogenous gene of the plant's genome to create an allelic variant, or allele of the gene. An induced mutant allele conferring glyphosate tolerance is also referred to herein as a “glyphosate tolerance mutant allele” or an “induced glyphosate tolerance mutant allele.” An induced mutant allele can be created in a wild-type endogenous gene, or in a variant or allele thereof. The induced mutant allele is stable and produces a heritable change in the phenotype of the plant carrying the allele, alone or in combination with other induced mutant alleles.

In certain embodiments, the glyphosate tolerant plant is free of recombinant glyphosate tolerance genes. As used herein, a “recombinant glyphosate tolerance gene” refers to a heterologous gene (i.e. a gene from a different, non-interbreeding family, genus or species) or a chimeric gene (i.e., a gene fusion comprising a heterologous gene operably linked to a chimeric promoter) that confers tolerance to glyphosate when introduced into the subject plant by genetic engineering methodologies. Methods of determining whether a plant has a recombinant glyphosate tolerance gene include, for example, using DNA hybridization, polymerase chain reaction, Northern hybridization, and the like (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein). For example, the polymerase chain reaction can be used to detect a chimeric promoter and/or terminator associated with a recombinant glyphosate tolerance gene. Plants carrying glyphosate tolerance mutant alleles, without a recombinant glyphosate tolerance gene, are referred to herein as “non-transgenic plants.” While these plants are non-transgenic with respect to the glyphosate tolerance trait, they can be transgenic or non-transgenic with respect to other traits or genes.

In some embodiments, a glyphosate tolerance mutant allele can be dominant. In other embodiments, a glyphosate tolerance mutant allele can be co-dominant, semi-dominant or possibly recessive. The phenotype conferred on a plant by a glyphosate tolerance mutant allele, or a combination of glyphosate tolerance mutant alleles, can be determined relative to a specified dosage of glyphosate.

The glyphosate tolerance mutant alleles can have dominant, semi-dominant, or possibly recessive inheritance (e.g., based on M3 generation segregation of progenies from tolerant M2 mutants). The glyphosate tolerance mutant alleles can be transmitted to progeny by plant breeding techniques (e.g., by self-pollination of mutants, by sexual recombination into F1 hybrids, or the like), which allows the transfer of these traits. Thus, the glyphosate tolerance mutant alleles can be introduced into, for example, commercial cultivars, varieties and experimental lines, by those skilled in the art of plant breeding.

As used herein, the term “glyphosate” includes any herbicidally-effective form of N-phosphonomethylglycine, including any of its several salts (e.g., the isopropylamine salt or the trimethylsulfonium salt), and other forms which result in the production of the glyphosate ion in plants. In some embodiments, the glyphosate is ROUNDUP ULTRAMAX® herbicide (Roundup® and Roundup Ultramax® are registered Trademarks of Monsanto).

Glyphosate tolerance mutant alleles can be present alone or in combination in a plant. The glyphosate tolerance mutant alleles can exhibit additive effects, such that different mutations can be genetically recombined in same plant to increase the level of tolerance. For example, the glyphosate tolerance mutant alleles, alone or in combination, can confer tolerance to dosage of glyphosate of at least 8 oz per acre, at least about 12 oz per acre, at least about 16 oz per acre, at least about 20 oz per acre, at least about 24 oz per acre, at least about 28 oz per acre, at least about 32 oz per acre, at least about 36 oz per acre, at least about 40 oz per acre, at least about 44 oz per acre, at least about 48 oz per acre, or at least about 52 oz per acre, or more, depending on the number of different glyphosate tolerance mutant alleles recombined. As used herein, “oz per acre” refers to the ounces applied per acre of a 50% solution of glyphosate (as the isopropylamine salt). In the field, the indicated dosage of glyphosate can be applied in a carrying volume of, for example, 10 to 20 gallons per acre. In the greenhouse, the indicated dosage of glyphosate can be applied in a volume of, for example, 80 gallons per acre.

Tolerance to a dosage of glyphosate refers to the ability of a plant to survive (i.e., the plant is not killed) by that dosage of glyphosate. In some cases, tolerant plants may temporarily yellow or otherwise exhibit some glyphosate-induced injury (e.g., excessive tillering and/or growth inhibition), but recover. Glyphosate tolerance also can be determined with respect to a wild-type plant of the same variety or cultivar. The reference (e.g., a wild-type or a non-mutant, normal genotype) plant can be of the same variety or cultivar (usually the non-mutagenized parent) recognizable by those skilled in the art as being susceptible to glyphosate.

In certain embodiments, a glyphosate tolerance mutant allele, alone or in combination, confers tolerance to elevated levels of glyphosate. As used herein, “elevated levels of glyphosate” refer to a dosage of at least about 12 oz per acre, at least about 16 oz per acre, at least about 20 oz per acre, at least about 24 oz per acre, at least about 28 oz per acre, at least about 32 oz per acre, at least about 36 oz per acre, at least about 40 oz per acre, at least about 44 oz per acre, at least about 48 oz per acre, at least about 52 oz per acre, or more.

In another aspect, methods are provided for modifying a plant's tolerance to glyphosate. In certain embodiments, glyphosate tolerance mutant alleles can be induced in plants susceptible to glyphosate (e.g., a “wild-type” or normal plant with respect to the glyphosate trait). A mutagenesis technology can be used to induce glyphosate tolerance mutant alleles of an endogenous plant gene(s). Further rounds of mutagenesis can be used by those skilled in the art to induce additional glyphosate tolerant mutant alleles in glyphosate tolerant target plants carrying glyphosate tolerant mutant alleles. As used herein, a “mutagenesis technology” refers to mutagenesis of a plant or plant part with a mutagen (e.g., a chemical or physical agent that increases the frequency of mutations in a target plant or plant part). In an exemplary embodiment, the double chemical mutagenesis technique of Konzak, as described in U.S. Pat. No. 6,696,294 (U.S. patent application Ser. No. 09/719,880, filed Dec. 18, 2000) and International Patent Publication WO 99/65292 (the disclosures of which are incorporated by reference herein), can be used to induce glyphosate tolerance mutant alleles in endogenous plant genes.

Glyphosate tolerance can be induced in a variety of plant species. As used herein, the term “plant” is intended to encompass plants at any stage of maturity or development. Plant parts include, but are not limited to, stems, roots, flowers, ovules, stamens, leaves, embryos, meristematic regions, callus tissue, anther cultures, gametophytes, sporophytes, pollen, microspores, protoplasts, seeds, and the like.

Suitable plant species for mutagenesis include both monocots and dicots. Suitable monocots include, for example, species in the orders Acorales, Alismatales, Arales, Arecales, Asparagales, Bromeliales, Commelinales, Cyperales, Dioscoreales, Hydatellales, Iridales, Juncales, Liliales, Orchidales, Pandanales, Poales, Typhales and Zingiberales. Exemplary monocots include, for example, banana, barley, corn (maize), sorghum (grain or forage types), oats, pineapple, rice, rye, wheat, onion, garlic, triticale, bluegrass (Poa pratensis), orchard grass, brome grass, perennial rye grass, bent grass, tall fescue and other fescues, Bermuda grass and other turf and forage grass species.

Suitable dicots include, for example, for example, species in the orders Amborellales, Apiales, Aquifoliales, Aristolochiales, Asterales, Austrobaileyales, Berberidopsidales, Boraginaceae, Brassicales, Buxales, Canellales, Caryophyllales, Celastrales, Ceratophyllales, Chloranthales, Cornales, Crossosomatales, Cucurbitales, Dilleniales, Dipsacales, Ericales, Fabales, Fagales, Garryales, Gentianales, Geraniales, Gunnierales, Lamiales, Laurales, Magnoliales, Malpighiales, Malvales, Myrtales, Nymphaeales, Oxalidales, Piperales, Proteales, Ranunculales, Rosales, Santalales, Sapindales, Saxifragales, Solanales, Trochodendrales, Vitales and Zygophyllales. In exemplary embodiments, the dicot can be, for example, cotton, lettuce, soybeans, spinach, sunflower, alfalfa, clover species, potatoes, tomatoes, bean and pea species (e.g., Vigna and Pisum) and rape, including Canola.

In a typical embodiment, the plant can be, for example, a crop plant such as wheat, rice, barley, triticale, maize (dent, semi-dent, flint, sweet corn, popcorn), sorghum (grain, forage), canola, carrots, coffee, cotton, dwarf beans, egg plant, forage crops, field beans, cow pea, flax, alfalfa, oat, oilseed rape, onions, peanuts, pea, pepper, perennial grass, potato, sweet potato, rice, rye, ryegrass, sorghum, soybean, sunflower, tea, tobacco, or the like. Suitable forage crops include, for example, wheatgrass, canarygrass, bromegrass, wild rye grass, forage sorghum, sudan grass, bluegrass, orchard grass, alfalfa, sanfoin, birdsfoot trefoil, medic, white lupine, alsike clover, red clover, white clover, and sweet clover. In other embodiments, the plant can be, for example, a cereal (e.g., barley, corn (maize), oat rice, rye, sorghum, triticale, wheat, millet, or the like), turf grasses, forage grasses, or the like. Suitable members of the grass family (Gramineae) include, for example, sorghum, rice, oat, wheat, triticale, rye, forage grasses (e.g., orchard grass), perennial Fescue grasses, bromegrass, lawn and greens grasses (e.g., Poa pratensis), or the like. Other suitable plants include, for example, Pigeon pea, lupini bean, lima bean, kenaf, cowpea and switchgrass. Suitable clovers (Trifolium spp.) include, for example, sweet clovers (white and yellow) and other Melilotus spp. Suitable members of the Solailaceae family include, for example, tomato, potato, Capsicum peppers, and eggplant. Suitable members of the Leguminoseae family include peas, peanuts, soybeans, cowpeas, and beans. Other suitable plants include vernonia (Vernonia anthelminica), safflower (Carthamus tinctorus L), pearl millet (Pennisetum americanum L), proso millet (Panicum miliaceum L.) and white mustard (Sinapis alba L.). The plant species can be diploid or polyploid plant species. As used herein, “polyploid” refers plant species comprising at least three sets of chromosomes or genomes (e.g., a triploid, a tetraploid, hexaploid or octaploid).

The plants can further include, for example, a cultivar, variety, breeding line or clone, including apomictic varieties, of any suitable plant species. The terms “cultivar” and “variety” refer to a group of plants within a species defined by the sharing of a common set of characteristics or traits accepted by those skilled in the art as sufficient to distinguish one cultivar or variety from another cultivar or variety. There is no implication in either term that all plants of any given cultivar or variety will be genetically identical at either the whole genome or molecular level or that any given plant will be homozygous at all loci. A cultivar or variety is considered “true breeding” for a particular trait if, when the true-breeding cultivar or variety is self-pollinated, more than 95% of the progeny carry the trait. Clonal reproduction assures reproduction of the same phenotype without sexual reproduction. Similarly, apomictic species, like bluegrass, buffel grass and buffalo grass reproduce their apomictic phenotypes at a nearly 100% rate, with little, if any, sexual reproduction.

The terms “breeding line” or “line” refer to a group of plants within a cultivar defined by the sharing of a common set of characteristics or traits accepted by those skilled in the art as sufficient to distinguish one breeding line or line from another breeding line or line. There is no implication in either term that all plants of any given breeding line or line will be genetically identical at either the whole genome or molecular level or that any given plant will be homozygous at all loci. A breeding line or line is considered “true breeding” for a particular trait if, when the true-breeding line or breeding line is self-pollinated, more than 95% of the progeny carry the trait.

As used herein, the terms “wheat” and “wheat plant” refer to a plant that is a member of the Triticum genus, including, but not limited to, T. aestivum, T. turgidum, T. timopheevi, T. dicoccoides, T. zhukovskyi, T. monococcum and T. urartu, and recombinants and hybrids thereof. Examples of T. aestivum subspecies include aestivum (common wheat), compactum (club wheat), macha (macha wheat), vavilovi (vavilovi wheat), spelta, and sphaerococcum (shot wheat). Examples of T. turgidum subspecies include turgidum, carthlicum, dicoccom, durum, paleocolchicum, polonicum, turanicum, and dicoccoides. Examples of T. monococcum subspecies include monococcum (einkorn), aegilopoides, and urartu.

In exemplary embodiments, the wheat plant is a hard red winter or spring wheat, a soft red winter wheat, a hard white spring or winter wheat, or a soft white spring or winter wheat, or the like. Typically, the wheat is cultivated variety or a breeding line.

As used herein, “triticale” and “triticale plant” refers to a plant that is created by crossing a rye plant (Secale cereale) with either a tetraploid wheat plant (e.g., Triticum turgidum or a hexaploid wheat plant (e.g., Triticum aestivum), followed by doubling the chromosomes to achieve a fertile and stable synthetic subspecies. Examples of triticale plants include, for example, X Triticosecale Wittmack (where X refers to the synthetic origin), cvs Jenkins, Juan, 102, Alzo, Presto, or the like.

Other exemplary plant species include, for example, Hordeum vulgare (six row barley), Hordeum disticum (two row barley), Triticum turgidum durum (all commercial durums), Triticum turgidum turanicum (a long kernel type durum), Avena sativa (hulled oat), Avena nuda (hulless oat), Oryza sativa ssp japonica (short grain, sticky rice; such as cv Calrose), Oryza sativa ssp indica (Indian type long grain, and Basmati types; cvs, such as Texmati); Oryza glabberima (African rice); and Zea mays (e.g., dent, semi-dent, flint, popcorn or sweet corn).

In another aspect, a plant's glyphosate tolerance can be altered by introducing an induced glyphosate tolerance mutant allele(s) into a target plant by plant breeding techniques. For example, a glyphosate tolerance mutant allele(s) can be introduced into a target cultivar, variety or line by plant breeding techniques. Such techniques also can be used to introduce multiple glyphosate tolerance mutant alleles into a target cultivar, variety or line, or to introduce (e.g., by crossing) additional glyphosate tolerant mutant alleles into a glyphosate tolerant target cultivar, variety, or line already carrying one or more glyphosate tolerant mutant alleles. The resulting progeny can be, for example, a desired glyphosate tolerant cultivar, variety or line used for commercial production and/or for research purposes. In addition, the resulting progeny can be intermediates in a breeding program.

In some embodiments, a plant's glyphosate tolerance can be increased or altered by introducing (e.g., by crossing) an induced glyphosate tolerance mutant allele(s) into a target plant comprising a recombinant glyphosate tolerance gene(s). The glyphosate tolerance mutant allele, or multiple glyphosate tolerance mutant alleles, can be introduced by plant breeding techniques. In certain embodiments, the resulting progeny plant is free of recombinant glyphosate tolerance genes. In other embodiments, the resulting progeny plant includes both the glyphosate tolerance mutant allele(s) and the recombinant glyphosate tolerance gene(s). The glyphosate tolerant phenotype of the progeny plant can be, for example, greater than the glyphosate tolerance phenotype of the target plant. The resulting progeny plants can be, for example, a desired glyphosate tolerant cultivar, variety or line used for commercial production and/or for research purposes. In addition, the resulting progeny plants can be intermediates in a breeding program.

In certain embodiments, glyphosate tolerance in a plant is due to the presence of an induced glyphosate tolerance mutant allele(s) in a plant (i.e., without contribution by a recombinant glyphosate tolerance gene(s)). In other embodiments, glyphosate tolerance is due to the presence of both an induced glyphosate tolerance mutant allele(s) and a recombinant glyphosate tolerance gene(s), wherein both the glyphosate tolerance mutant allele(s) and recombinant glyphosate tolerance gene(s) contribute to the glyphosate tolerant phenotype.

In additional aspects, progeny plants, plant cells, and seed may be produced from or by glyphosate tolerant plants; A progeny plant, plant cell and plant seed may carry a glyphosate tolerance mutant allele or multiple glyphosate tolerance mutant alleles. A progeny plant can be derived from a glyphosate tolerant plant as a direct, first generation descendent or indirectly, as a descendant of an ancestor glyphosate tolerant plant. Seeds according to the present invention can be from a glyphosate tolerant plant or from the progeny of such plants. In certain embodiments, the seed is true breeding for glyphosate tolerance.

In another aspect, methods of controlling weeds within the vicinity of a glyphosate tolerant plant are provided. The methods comprise applying glyphosate to the weeds and optionally to the glyphosate tolerant plant, wherein the glyphosate tolerance of the plant is due to the presence of a glyphosate tolerance mutant allele(s) in the plant genome. The glyphosate typically kills the weeds. In a related aspect, methods of controlling weeds in a field are provided. The methods comprise applying glyphosate to the field, after emergence of the glyphosate tolerant plants and growth to the three to five leaf stage, wherein the glyphosate tolerance of the plant is due to the presence of a glyphosate tolerance mutant allele(s) in the plant genome. The glyphosate typically kills only the weeds.

The glyphosate tolerant plants can be, for example, crop plants (e.g., agronomic, vegetable, turf grass or horticultural plants). The plants can be, for example, alfalfa, beans, bent grass, bermuda grass, blue grass, brome grass, cereal, carrot, chickpea, cotton, cowpea, cucumbers, dwarf bean, fescue, field bean, flax, forage grasses, garlic, kenaf, lima bean, lupini bean, oilseed rape, onion, peas, peanut, peppers, pigeon pea, pineapple, potato, ryegrass, soybean, squash, sugar beets, sunflower, tomato, or the like. The cereal can be, for example, barley, corn, millet, oats, rice, rye, sorghum, triticale, wheat, or the like. The wheat can be, for example, a bread wheat or durum wheat. In some embodiments, the glyphosate tolerant plant is free of recombinant glyphosate tolerance genes. In other embodiments, the glyphosate tolerant plant comprises a glyphosate tolerance mutant allele(s) and a recombinant glyphosate tolerance gene(s).

Glyphosate can be applied, for example, at a dosage of at least 8 oz per acre, at least about 12 oz per acre, at least about 16 oz per acre, at least about 20 oz per acre, at least about 24 oz per acre, at least about 28 oz per acre, at least about 32 oz per acre, at least about 36 oz per acre, at least about 40 oz per acre, at least about 44 oz per acre, at least about 48 oz per acre, or at least about 52 oz per acre, or more.

The weeds can be, for example, annual grass, biennial grass, perennial grass, broadleaf weeds, and volunteer crop plants; such weeds include, but are not limited to, wild oats, foxtail grasses, quackgrass, pigweed, field bindweed, wild buckwheat, knapweed, cheat grass, Barnyard grass, goat grall, black grass, sweet clover, smartweed, yellow mustard, kochia, cocklebur, velvet leaf, wild sunflower, biennial wormwood, and/or Russian thistle. The methods can optionally further include, for example, growing the plants, harvesting the seed, and/or replanting the seed.

The following examples are provided merely as illustrative of various aspects of the invention and shall not be construed to limit the invention in anyway.

### EXAMPLES

### Example 1

Glyphosate tolerant wheat plants were isolated by chemical mutagenesis. These wheat plants carry one or more induced glyphosate tolerance mutant alleles.

Procedures

Mutagenesis: The mutagenesis of wheat seeds, for the induction glyphosate tolerance mutant alleles, was performed according to the method of Konzak, as disclosed in U.S. Pat. No. 6,696,294 (U.S. patent application Ser. No. 09/719,880, filed Dec. 18, 2000) (the disclosure of which is incorporated by reference herein).

The mutagenesis procedure was as follows: approximately 3 kg of wheat seeds of each wheat variety or line were presoaked in a container with local tap water for about 6 hours at room temperature (about 72° F.). After presoaking, the tap water was replaced with distilled water, the seeds were placed in 2-4 L plastic containers, and 3 mls or 3.5 mls/L of ethyl methanesulfonate (EMS) mutagen was added. The treatments were conducted in a fume hood. The seeds were allowed to imbibe the mutagen solution for 2 hours at room temperature, after which the EMS solution was poured off into a disposal container, and 1 liter of 0.001 M phosphate buffer at pH 3.5 was added to the seeds. Then, 2 ml/L of a 1 M sodium azide (AZ) solution was added to each treatment container, and allowed to be imbibed for one hour. The containers with seeds being treated were shaken periodically for 10-15 seconds about every 10 minutes during the treatment periods with each mutagen, to assure a uniform distribution of the chemical mutagens among the seeds. Following the azide treatments, the chemical solutions were poured off into a disposal container, the seeds were then rinsed twice with tap water, and spread out to dry on paper towels in plastic trays. The seeds were allowed to redry for about 48 hours at room temperature to a moisture content of about 15% moisture.

The re-dried, mutagenized seeds were transferred to planting trays, and sown in a field for production of the M1 generation plants, which produced M2 (second generation) seed by self-pollination. At maturity, the M1 plants (with M2 seeds) were harvested in bulk. The M2 seeds were stored in a dry area, cleaned of debris through an air cleaner, and held in storage until planting for the screening trials was done.

The screening trials of M2 progenies were conducted at two locations: Warden, and Pullman, Washington (abbreviated as “W” or “T”, respectively, in Table 1). At Pullman, five or more varieties/lines/genotypes were combined in a bulk mix made up of M2 mutagenized seeds. At Warden the seed lots were sown by individual variety/line.

The seed lots were sown in strips, one drill width wide (approximately 6 feet) per pass up and down the field, with a small cereal grain drill. The different M2 seed lots were sown, one following another, until each entire seed lot was used. The field at Warden was approximately 4 acres in size, in a rectangular shape, and irrigated by sprinklers. Glyphosate (50.2% active ingredient, glyphosate, N-(phosphonomethyl)glycine isopropylamine salt; 49.8% other ingredients; Monsanto ROUNDUP ULTRAMAX® was applied when the plants were at the three to five leaf stage (about 4-5 weeks after the seed was sown). The field was sprayed once with a dosage of 16 oz per acre application of glyphosate (50% commercial product), and after another 4 weeks a few plants surviving the treatment were dug and transferred to a greenhouse in Pullman for further growth, evaluation and testing. Glyphosate was applied in 20 gallons per acre volume in the field or 80 gallons per acre volume in the greenhouse.

Because there were unsprayed sections of the field that were missed in the first herbicide application, a second 16 oz per acre glyphosate spray was applied over the field at about 7-9 leaf stage. Again, about 4 weeks later a small number of surviving plants were dug and transferred to the greenhouse at Pullman for further growth, evaluation and testing. Selected plants could have been allowed to mature in the field, and the M3 seed harvested for subsequent screening. Within a few weeks after the plants were transferred to the greenhouse, they appeared to have recovered from their move, and all were sprayed with glyphosate at 40 oz per acre applied in the equivalent of 80 gallons water per acre, inside a chamber designed for herbicide spray application to small lots of plants. Some selections from the field were tolerant to the applied glyphosate dose, but the majority of plants proved susceptible in the greenhouse test, and were considered “escapes.” Following the spray treatments, the plants were allowed to increase in size.

Samples of plant tissue were taken for DNA analyses (infra). Thereafter, the plants (all winter habit) were placed in a cold chamber for a 2 month vernalization treatment period, after which they were returned to the greenhouse and allowed to produce seed for progeny tests.

A second lot of M2 mutagenized wheat was sown in a small field area outside of Pullman, Washington. This lot of plants included the M2 stock of a hard red spring (HRS) wheat experimental line NPBM00505, as well as M2 seed of two experimental spring durums and the M2 of two Northwest Plant Breeding soft white winter wheats. After the seedlings emerged and had grown to the three to five leaf stage, an application of 20 oz per acre glyphosate was applied to the developing seedling plants in the field. About three to four weeks after the herbicide was applied, a small number of surviving plants. (approximately 32) were dug, transferred to greenhouse pots, and allowed to develop in the greenhouse. After about two weeks recovery from the field transfer, all selected plants were sprayed with a 40 oz per acre dose of glyphosate herbicide (about 50% commercial product concentrate). About two weeks later, leaf samples were taken for DNA analyses.

The plants were allowed to continue their growth and development through to seed production. The winter wheat selections were transferred to a 6° C. cold chamber for vernalization over a 2 month period. The spring wheat plants were allowed to develop and produce seed for progeny analyses of their herbicide tolerance traits. The winter wheat plants were allowed to develop for seed production after their vernalization treatment period.

The HRS wheat plants produced a rather large quantity of seed that proved to have an after-ripening dormancy. To overcome this dormancy, the seeds were initially started in Petri dishes with a treatment (a nitrogen source (sodium nitrate) and kinetin) to help break the dormancy, including germinating them in a refrigerator at 4° F. for about 1-2 weeks, after which the seeds showed evidence of germination by their exposed shoot apices. The germinating seeds were transferred to small greenhouse trays for growing to their 3-5 leaf stage, at which point they were sprayed with glyphosate solutions made with a commercial 50% glyphosate product, as was used in the field screening study (see Table 1).

DNA Analyses: Samples of plant tissue were taken for recombinant glyphosate tolerance testing (GMO analysis). The samples were tested for the presence of the NOS terminator and 35s promoter sequences by polymerase chain reaction (PCR). The NOS terminator and 35s promoter sequences are present in the Roundup Ready™ wheat and maize plants that carry the genetically engineered glyphosate tolerance trait. The Pullman and Warden samples proved negative for the GMO markers (Table 1).

Results

Glyphosate tolerant plants were isolated from hard red and hard white spring wheats and hard red, hard white and soft white winter wheats. Glyphosate tolerant plants of each variety were tested to confirm genetic inheritance and transmissibility of the glyphosate tolerant trait.

The glyphosate tolerant plants were tolerant to doses of 8 oz., 16 oz, 24 oz and/or 32 oz per acre. In addition, certain glyphosate tolerant wheat plants exhibited tolerance to doses up to at least 40 oz per acre of glyphosate.

The glyphosate tolerant plants were progeny tested. The mutant alleles were shown to be heritable and dominant, semi-dominant or possibly recessive. For example, the progeny from three M2 HRS wheat mutants (see Table 1: 8s, 9s, 10s (infra)) appear to carry at least one homozygous glyphosate tolerant mutant allele (i.e., the M3 progeny of wheat mutants HRS00505-8s, 9s and 10s were mostly tolerant to an 8 oz dose of glyphosate). Progeny from other M2 (heterozygous) mutant plants showed genetic segregation ratios of 3:1, or for two mutant plant progenies, a 15:1 ratio of tolerant to non-tolerant plants, when sprayed with an 8 oz per acre dose of glyphosate. (Seed from glyphosate tolerant progeny of 9s, designated gT-9s, was deposited with the American Type Culture Collection on Dec. 21, 2004 as ATCC Deposit No. PTA-6482.)

In some cases, the glyphosate tolerant plants initially showed leaf yellowing, characteristic of glyphosate sensitivity but later produced green tillers and continued to develop normally. Some other seedlings showed growth inhibition and excessive tillering, but also recovered. The glyphosate tolerant plants exhibiting a 15:1 ratio demonstrated that, in fact, two independently inherited mutant loci were induced in the same embryonic cell by the mutagenic treatments applied to the seeds. This may have resulted from the induction of two independent mutations in the same cell of the seed embryo as a consequence of the method by which the two chemical mutagens were tandemly applied to the seeds.

For the glyphosate tolerant spring wheat plants, a number of crosses were made to non-tolerant wheats using pollen from the tolerant plants. These genetic analyses were initiated to confirm inheritance and transmissibility of the glyphosate tolerance trait, or traits, in each separate mutant plant. Progeny testing was initiated following maturation of the seed.

For the hard red spring line, seed was produced in the greenhouse at Pullman. As soon as the harvested seeds would germinate, the M3 generation seed progeny of each selected plant line were sown in greenhouse trays for evaluation of their tolerance to an 8 oz per acre application of the herbicide.

In further studies of the glyphosate tolerant mutant wheat plants, crosses have also been made to transfer the tolerance trait(s) to non-tolerant genetic backgrounds. The results from M3 progeny tests showed that the tolerance trait(s) exhibits Mendelian inheritance, although in many cases multiple glyphosate tolerance mutant alleles may be present in the same progeny. In such cases, the progeny appear to exhibit dose dependent glyphosate tolerance, presumably depending on the number and nature of glyphosate tolerance mutant alleles inherited by each progeny.

In a test of M3 generation progeny from one plant, NPBM00505-14s, six plants were sprayed with a 40 oz per acre dose. While four of the six plants succumbed to the application of 40 oz per acre, two plants recovered after showing some yellowed shoots, and then produced normal green tillers that continued to grow. The plants eventually produced fertile spikes. Leaf samples from the two surviving plants of NPBM00505-14s were taken and subjected to DNA analyses. The results confirmed that their glyphosate tolerance was not due to contamination with GMO germplasm (i.e., containing a recombinant gene).

Other M3 progeny from M2 plant NPB00505-14s were then grown for a larger scale set of herbicide spray treatments. Progeny from this plant (14s), sprayed with an 8 oz per acre dose of glyphosate, segregated for tolerance in a ratio of 15 tolerant to 1 non-tolerant (susceptible), indicating that the selected M2 plant carried two independent glyphosate tolerance loci (Table 1).

Further tests were initiated in an effort to identify plants carrying both of the mutations identified by the 8 oz per acre glyphosate application. Three flats of progeny from the 14s plant were sprayed with herbicide applications either of 16 oz or 32 oz per acre doses of herbicide. Analyses from these tests indicated that the tolerance levels provided by the two mutant loci interact, contributing additively to herbicide tolerance, each mutant locus contributing tolerance to the 16 oz per acre dose of glyphosate, according to a segregation frequency of 9 tolerant to 7 non-tolerant seedlings. Tolerance to the 32 oz dose per acre was achieved by the additive interaction of the mutant alleles, in accordance with a 7 tolerant to 9 non-tolerant seeding ratio. Neither of the two mutations, even when homozygous by themselves, appears to provide tolerance to the 32 oz per acre dose, but when one of each allele was present in the heterozygote, tolerance was provided to a 32 oz per acre dose, due to the interaction of the glyphosate tolerance mutant alleles as independent dominantly inherited mutant loci.

The results from the tests of several mutant spring wheat progenies shown in Table 1, confirmed the tolerance of the M2 plants, also via their progeny, demonstrating that the induced mutant tolerance is stably inherited and controlled by one or two independently-induced mutant alleles. Also, mutants present in the progeny of different wheat varieties must be due to independent mutational events. Such mutants could represent different mutant alleles of loci located in one or more of the A, B and/or D genomes of the hexaploid wheats. In summary, analyses of progeny of one mutant HRS wheat plant indicates that two mutations conferring near equal, independent and additively interactive levels of tolerance to the 8 oz per acre and 16 oz per acre concentrations of glyphosate were induced.

TABLE 1
Characteristics of Glyphosate Tolerant Plants

| | Presence | | | |
|---|---|---|---|---|
| Sample | of GMO | Source of | | |
| No. | DNA¹ | M2 Plant | Results | Dose |
| 1sww (winter) | — | W | 33T, 5S, 15 HT | 16 oz/acre |
| 1ww (winter) | — | W | 8T, 25S, 1HT | 32 oz/acre |
| 2swp (winter) | — | P | 2T, 8S, 12HT | 32 oz/acre |
| 2ww (winter) | — | W | 3T, 0S, 44HT | 16 oz/acre |
| | | | | 2X² |
| 2ww (winter) | — | W | 15T, 29S, 22HT | 32 oz/acre |
| 3ww (winter) | — | W | 13T, 11S, 10HT | 32 oz/acre |
| 5swp (winter) | — | P | 5T, 29S, | 32 oz/acre |
| 7swp (winter) | — | P | 13T, 23S | 32 oz/acre |
| 8s (spring) | — | P | 84T, 1S | (8 oz/acre) |
| 8s (spring) | — | P | 78T, 1S, 2HT | 8 oz/acre |
| 8s (spring) | — | P | 3T, 21S | 32 oz/acre |
| 8s (spring) | — | P | 4T, 16S | 40 oz/acre |
| 9s (spring) | — | P | 26T, 0S | 8 oz/acre |
| 9s (spring) | — | P | 10T, 5S | 8 oz/acre |
| 9s (spring) | — | P | 38T, 9S | 40 oz/acre |
| 9s (spring) | — | P | 50T, 5S | (8 oz/acre) |
| 10s (spring) | — | P | 69T, 3S | 8 oz/acre |
| 10s (spring) | — | P | 89T, 4S | (8 oz/acre) |
| 13s (spring) | — | P | 13T, 8S, 6S/R, | 32 oz/acre |
| | | | 17HT | |
| 14s (spring) | — | P | 2T, 8S | (8 oz/acre) |
| 14s (spring) | — | P | 72T, 3S, 5S/R | (8 oz/acre) |
| 14s (spring) | — | P | 1T, 0S | (8 oz/acre) |
| 14s (spring) | — | P | 19T, 19S, 6S/R | (16 oz/acre) |
| 14s (spring) | — | P | 9T, 33S | (32 oz/acre) |
| 15s (spring) | — | P | 1T, 4S | (8 oz/acre) |
| 16s (spring) | — | P | 0T, 3S | (8 oz/acre) |
| 17s (spring) | — | P | 29T, 1S, 8S/R | (8 oz/acre) |
| 17s (spring) | — | P | 6T, 1S | (8 oz/acre) |
| 17s (spring) | — | P | 39T, 0S, 2S/R | 8 oz/acre |
| 18s (spring) | — | P | 89T, 1S, 2S/R | (8 oz/acre) |
| 18s (spring) | — | P | 23T, 0S | (8 oz/acre) |
| 19swp (winter) | — | P | 0T, 43S | 32 oz/acre |
| 20swp (winter) | — | P | 21T, 16S | 32 oz/acre |
| 21s (spring) | — | P | 95T, 19S | (8 oz/acre) |
| 21s (spring) | — | P | 2T, 0S | (8 oz/acre) |
| 21s (spring) | — | P | 95T, 4S, 0S/R | 8 oz/acre |
| 21s (spring) | — | P | 43T, 0S, 8S/R | 8 oz/acre |
| 22s (spring) | — | P | 34T, 2S, 8S/R | 8 oz/acre) |
| E1 (winter) | — | W | 47T, 0S | 16 oz/acre |
| E1 (winter) | — | W | 13T, 4S, 23HT | 32 oz/acre |
| E1 (winter) | — | W | 2T, 5S, 1S/R | 40 oz/acre |
| | | | | 3X³ |
| E2 (winter) | — | W | 8T, 34S | 40 oz/acre |
| 1d (durum) | NYT | P | 45T, 90S | 16 oz/acre |
| 17d (durum) | NYT | P | 5T, 0S, 2HT | 16 oz/acre |
| 24d (durum) | NYT | P | 6T, 15S, 3HT | 16 oz/acre |
| 33d (durum) | NYT | P | 5T, 8S, 0HT | 16 oz/acre |
| 35d (durum) | NYT | P | 11T, 20S, 0HT | 16 oz/acre |
| 35d (durum) | NYT | P | 5T, 26S, 2HT | 32 oz/acre |
| 36d (durum) | NYT | P | 5T, 10S, 10HT | 16 oz/acre |
| RR Positive | + | N/A | | |
| Control | | | | |
| ¹PCR test for 35S and NOS sequences. | | | | |
| ²Sprayed twice. | | | | |
| ³Sprayed three times. | | | | |
| “P” denotes Pullman location; | | | | |
| “W” denotes Warden location; | | | | |
| “T” denotes tolerant; | | | | |
| “S” denotes susceptible; | | | | |
| “HT” denotes tolerant, but “high tillered”; | | | | |
| “S/R” denotes susceptible, but recovered; | | | | |
| “RR Positive Control” denotes a ‘Roundup Ready ® plant (Roundup Ready ® is a registered trademark of Monsanto); | | | | |
| “NYT” means tests not done. | | | | |
| Note: | | | | |
| In some tests, the 8 oz per acre dose was too low to cause plant death, because many plants classified as susceptible later recovered from the herbicide application, and produced green shoots. Thus, these plants were initially scored according to the initial susceptibility of the plants (S/R designation in Table 1). The results suggest that for certain varieties or lines, a 16 oz per acre dose of glyphosate may be the minimum | | | | |
| # required for ready differentiation of plants susceptible to glyphosate versus those that are tolerant. Most mutant alleles identified provide tolerance to at least a 16 oz per acre dose rate, while some mutants are tolerant to a 24 oz per acre rate (M4 screening data), 32 oz per acre rate or 40 oz per acre rate, or may be tolerant to a higher dosage of the herbicide. | | | | |

### Example 2

Glyphosate tolerant mutant alleles can be transferred to non-tolerant wheat plants. Glyphosate tolerant mutant alleles in SWW, as in M2 ELTAN (=ME2) are transferred by backcross to ELTAN by generating double haploid (DH) lines from F1 hybrids [M2 ELTAN×ELTAN] (DH—U.S. Pat. No. 6,764,854; the disclosure of which is incorporated by reference herein) or by making F2 lines. The lines are tested for tolerance to a 16 oz, 32 oz, 40 oz, or 52 oz per acre dose of glyphosate.

| Parent 1 | Parent 2 | Method |
|---|---|---|
| HWW-ME1 | HWW = NPB-M2WW | DH or F2 |
| HWS, = Klasic/Platte lines | NPB00505-M9s | DH or F2 |
| HRS = NPB00505-9s | WED202-16-2 | DH or F2 |
| HWW = NPBHWW-ME1 | NPB00004HWW | DH or F2 |
| HWW = NPBHWW-ME1 | NPB-M1WW | DH or F2 |
| HWW = NPBHWW-ME1 | Eltan/NPB-ME2 F1 | DH or F2 |
| HWW = NPBHWW-ME1 | NPB-M2WW | DH or F2 |

### Example 3

Glyphosate tolerant mutant alleles are recombined in progeny plants. Glyphosate tolerant plants of NPB00505-M9s and NPB00505-M8s can be combined by crossing to form an F1 from which double haploid (DH) lines are produced, which are then tested for tolerance to a 16 oz, 32 oz, 40 oz, or 52 oz., dose of glyphosate.

Other combinations of glyphosate tolerant mutant alleles can be isolated as follows:

| Parent 1 | Parent 2 | Method |
|---|---|---|
| NPB00505-M9s | NPB00505-M13s | DH or F2 |
| NPB00505-M9s | NPB-ME1 | DH or F2 |
| NPB-ME1 | NPB-ME2 1WW | DH or F2 |
| NPB-ME2 | 20 SWWP | DH or F2 |
| NPB-ME2 | ELTAN | DH or F2 |
| NPB-ME2 | HWW = NPB-E1 | DH or F2 |
| NPB-ME2 | NPBMSWWXX | DH or F2 |

### Example 4

Glyphosate tolerant varieties of dicotyledonous species (dicots) can be prepared according to the following description. Briefly, the mutagenesis procedure of Konzak (U.S. Pat. No. 6,696,294), with the following variation, was used in order to reduce imbibition damage to the treated seeds and enhance germination of the mutagenized seed. Dicot seeds are given a priming pretreatment (sometimes termed ‘matriconditioning’ (Khan, et al., Crop Science 32: 231-7 (1992)). 1000 g Celite™ or Kenite™ (diatomaceous earth) are mixed with 3250 ml of water, and subsequently 500 g. seeds are mixed in. The mixture is placed in a large container (e.g., a plastic 1 gallon bottle) and rotated for about 18 to about 36 hours at about 65° F., or by mixing every 4-6 hours for the priming period. Sufficient space in the bottles is allowed, so that the mixture of seeds and powder will flow and continuously mix as the bottles are rotated. After the priming treatment, the seeds are removed from the diatomaceous earth by sieving, and then rinsed with water over a period of about 2 minutes. Then about 3 liters of seeds and 3 liters of distilled water are combined in containers, the volume of water being sufficient to just cover the seeds in each container. EMS (ethylmethane sulfonate) is added to a concentration of about 2.0-4.0 ml per liter, and the containers are gently shaken to mix the seeds with the mutagen solution. The mixture is gently shaken again each 10 minutes, for at least 10-15 seconds, during a 2 hour treatment period. After the mutagenesis treatment, the EMS solution is decanted off into disposal containers, with sodium thiosulfate added to degrade the EMS.

Following the EMS treatment, three liters of phosphate buffer (monobasic NaHPO₄, adjusted to pH 3.0-3.5) is added to cover the seeds. Then 2 ml per liter of a 1 M stock solution of sodium azide is added per liter of buffer to each bottle, irrespective of EMS dose. The mixture is shaken repeatedly over a 1 hour period, for about 10-15 seconds per each shaking. After the azide treatment for 1 hour, the azide solution is poured off into a disposal container, and the seeds are then rinsed with tap water 2-3 times over 4-5 minutes. If desired, the mutagen-treated seeds may be treated with a fungicide (Captain™ or the like). The seeds are then placed onto a sieve to allow excess water to drain, placed in about 5 times the seed volume of dry diatomaceous earth (Kenite™) and mixed to remove all excess water. If the amount of diatomaceous earth used is not enough to absorb the free water, or if the diatomaceous earth powder is noticeably wet, the seeds are sieved to remove the moist powder, and fresh dry diatomaceous earth powder is used to incorporate with the treated seeds.

The seed/powder mixture is then spread out on cotton or burlap cloth covered trays, leaving only a small amount of powder (3 times seed volume) covering the seeds so that the moisture will evaporate. To assist re-drying, a fan can be placed to blow over the seeds. After about 24-36 hours with occasional re-mixing, the seeds will be dry enough to begin testing the seed viability. The powder is then shaken off the seeds using a sieve screen smaller than the seeds, and the seeds are placed in a tray to further dry in a moderately cool room (e.g., about 65-70° F. on a greenhouse bench). Planting of the seeds should be done as soon as possible after the mutagen treatments have been completed

After redrying sufficiently, the seeds are planted in soil to produce the M1 generation of mutagenized plants, which produce M2 generation seeds. The M2 generation seeds are then planted to produce M2 plants, which are sprayed with the herbicide glyphosate after the plants reach sufficient growth (typically the 3-5 leaf stage). After allowing a period of time for the herbicide to kill the major population, the field of M2 bulk progeny plants is screened to identify putative tolerant plants, which can be dug and transferred to a greenhouse for growth to produce seed or allowed to mature in the field.

After the transferred seedlings have recovered from the transfer to the greenhouse, they can be given a retest spray of herbicide to confirm their tolerance, then allowed to produce seed. The resulting seed can be used to confirm their herbicide tolerance and to produce a population of progeny to determine the genetic segregation of tolerance, and reconfirm their herbicide tolerance. Tolerant plants are repotted to continue their growth to produce seed, which can then be used for inter-crossing among different mutants to increase the level of tolerance by additive action of the mutant genes. Usually at least one backcross is desirable to remove possible secondary mutations from among the segregants. The mutants can then be used, for example, to develop varieties or lines for further use or commercialization.

### Example 5

For self-pollinating species, the mutagenesis technique for wheat, as described by Konzak (U.S. Pat. No. 6,696,294), or as modified in Example 4, can be used. Suitable species include, for example, barley, oats, triticale, sorghum, Canola, soybeans, and other legumes and certain grasses. The method described in Example 4, is particularly useful to retain seed viability of dicot species. Usually at least one backcross is desirable to remove possible secondary mutations from among the segregants.

### Example 6

For cross-pollinating monocotyledonous species, the mutagenesis technique for wheat, as described by Konzak (U.S. Pat. No. 6,696,294), or as modified in Example 4, can be used. Suitable species include, for example, corn (maize), and certain grasses. The mutagenized M1 generation plants are allowed to cross-pollinate naturally, but the population must be grown in an area isolated from other compatible species/varieties. The M2 seedlings can be screened in the field much as done for wheat. Usually at least one backcross is desirable to remove possible secondary mutations from among the segregants.

### Example 7

For apomicts, the mutagenesis technique of Example 4 can be used. The M1 generation of progeny are grown to produce MA2 seed. The MA2 seeds are sown in a field, and after the plant growth is sufficient, they are sprayed with at least a 16 oz dose of glyphosate. Tolerant plants are then dug and transferred to a greenhouse for confirmation tests and growth to produce seed. The confirmed tolerant plants are grown out to produce seed, which should be proved to show no segregation for tolerance. The seed can be used for further multiplications, and if tolerance is adequate release into commerce.

### Example 8

For vegetable species, radish, eggplant, lettuce, and the like (including Canola and rapeseed), the seeds can be mutagenized as in the modified method using diatomaceous earth, as in Example 4. The amount of EMS can be adjusted. The treated seeds are grown to produce an M2 population, which is then sown to screen for tolerance to the herbicide. Mutants with sufficient tolerance can then be used as parents for variety development. Usually at least one backcross is desirable to remove possible secondary mutations from among the segregants. The selected plants can be used for increase and commercialization or for further breeding to develop tolerant varieties.

### Example 9

Inbred lines, such as of maize, sorghum, onion, carrot, parsnip, or the like, can be mutagenized much as described for wheat, or as in Example 4, and tolerant plants isolated as described for wheat. Usually at least one backcross is desirable to remove possible secondary mutations from among the segregants. The tolerant selections can then be used as parents to transfer the trait to the inbred lines of the hybrid using a backcross procedure.

### Example 10

For sugar beets (for which the commercial varieties are often triploids), dominant glyphosate tolerant mutant alleles can be isolated in the tetraploid lines. The resulting tolerant plants can then be the parents for many varieties. Once a tolerant selection is identified or bred by recombination breeding among mutants, the tetraploid line can then be used to develop triploid varieties using any diploid line as a non-tolerant parent.

### Example 11

For other species for hybrids, such as carrot, onion, tomato or the like, mutants are produced much as described in example 4. The selected mutants are used to transfer the trait to the inbred lines. If preliminary tests show dominance of the trait to be sufficient, then only one of any two inbred lines need carry the tolerance, since the F1 hybrid will then be tolerant.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

1-16. (canceled)

17. A polyploid plant comprising an induced mutant allele of an endogenous gene that confers tolerance to glyphosate, as compared with a wild-type plant of the same species, wherein the glyphosate tolerance is due to the presence of the induced mutant allele of the endogenous plant gene.

18. The polyploid plant of claim 17, wherein the polyploid plant is free of recombinant glyphosate tolerance genes.

19. The polyploid plant of claim 17, which is a cereal.

20. The polyploid plant of claim 19, wherein the cereal is a triticale or wheat plant.

21. The polyploid plant of claim 20, wherein the wheat is a T aestivuin, T turgidum, T. timopheevii, T. zhukovslzyi, species or a hybrid thereof.

22. The polyploid plant of claim 21, wherein the wheat is a bread wheat or durum wheat.

23. The polyploid plant of claim 17, comprising at least two different induced mutant alleles in different endogenous genes, each mutant allele conferring tolerance to glyphosate.

24. The polyploid plant of claim 23, wherein the induced mutant alleles are in different genomes.

25. Seed derived from the polyploid plant of any one of claims 17 to 24.

26. A progeny plant derived from the polyploid plant of claim 17.

27. Seed derived from the progeny plant of claim 26.

28. The plant of claim 17, comprising tolerance to a dosage of glyphosate of about 8 oz per acre.

29. The plant of claim 28, comprising tolerance to a dosage of glyphosate of about 16 oz per acre.

30. The plant of claim 29, comprising tolerance to a dosage of glyphosate of about 24 oz per acre.

31. The plant of claim 30, comprising tolerance to a dosage of glyphosate of about 32 oz per acre.

32. The plant of claim 31, comprising tolerance to a dosage of glyphosate of about 40 oz per acre.

33. The plant of claim 32, comprising tolerance to a dosage of glyphosate of about 52 oz per acre.

34. A polyploid wheat plant comprising an induced mutant allele of an endogenous wheat gene that confers tolerance to glyphosate, wherein the wheat plant is free of recombinant glyphosate tolerance genes.

35. The polyploid wheat plant of claim 34, comprising tolerance to a dosage of glyphosate of about 8 oz per acre.

36. The polyploid wheat plant of claim 35, comprising tolerance to a dosage of glyphosate of about 16 oz per acre.

37. The polyploid wheat plant of claim 36, comprising tolerance to a dosage of glyphosate of about 24 oz per acre.

38. The polyploid wheat plant of claim 37, comprising tolerance to a dosage of glyphosate of about 32 oz per acre.

39. The polyploid wheat plant of claim 38, comprising tolerance to a dosage of glyphosate of about 40 oz per acre.

40. The polyploid wheat plant of claim 39, comprising tolerance to a dosage of glyphosate of about 52 oz per acre.

41. The polyploid wheat plant of claim 34, wherein the wheat is a bread wheat or a durum wheat.

42. The polyploid wheat plant of claim 34, comprising at least two different induced mutant alleles in different endogenous wheat genes that confer tolerance to glyphosate.

43. The polyploid wheat plant of claim 42, comprising two different induced mutant alleles in different endogenous genes, each of the mutant alleles in a different EPSPS gene.

44. The polyploid wheat plant of claim 42, each of the induced mutant alleles in a different wheat genome.

45. Wheat seed derived from the plant of any one of claims 34 to 44.

46. A progeny wheat plant derived from the plant of any one of claims 34 to 44.

47. Seed derived from the progeny wheat plant of claim 46.

48. A wheat plant comprising an induced mutant allele of an endogenous gene, the induced mutant allele conferring tolerance to glyphosate as compared with a wild-type wheat plant, wherein the glyphosate tolerance is due to the presence of the induced mutant allele derived from one of the following glyphosate tolerant lines: NPB00505-8s, NPB00505-9s, NPB00505-10s, NPB00505-13s, NPB00505-14s, NPB00505-17s, NPB00505-18s, NPB00505-21s, NPB-E1, NPB-1WW, NPB-2WW NPB-E2 or NPB-26WW.

49-90. (canceled)

91. A method of altering the glyphosate tolerance of a target plant, comprising:
crossing the plant of claim 34 with a target plant to form a progeny plant having a glyphosate tolerant phenotype, the progeny plant comprising the glyphosate tolerance mutant allele.

92. A method of controlling weeds within the vicinity of a crop plant comprising:
applying glyphosate to weeds and the plant of claim 34 which comprises at least one induced mutant allele conferring increased tolerance to the glyphosate as compared to a wild-type variety of the plant.
